(19) United States
(12) Patent Application Publication
(10) Pub. No.: US 2012/0177576 A1
(43) Pub. Date: Jul. 12, 2012

(54) OPTICAL DEVICE AND METHOD FOR NON-INVASIVE REAL-TIME TESTING OF BLOOD SUGAR LEVELS

(76) Inventor: Jun Jack Hu, Fairlawn, OH (US)

(21) Appl. No.: 13/496,987

(22) PCT Filed: Sep. 17, 2010
(86) PCT No.: PCT/US10/02531

§ 371 Date: Mar. 19, 2012

Related U.S. Application Data
(60) Provisional application No. 61/277,021, filed on Sep. 18, 2009.

(51) Int. Cl. A61K 49/00

(52) U.S. Cl. 424/9.1

(57) ABSTRACT

A device and method for non-invasive real-time testing of blood sugar levels in a diabetic patient. Specifically, this invention is directed to an optical device comprising a contact lens having a glucose-sensing optical pattern imprinted, marked, coated or otherwise disposed on or incorporated within the contact lens. The indicator pattern is further comprised of a glucose-sensing coating containing a boronic acid derivative, which reacts in the presence of glucose to create a readable pattern, which can then be correlated to a pre-determined or pre-calibrated blood glucose level. A polarized light source is one method that may be used to read the indicator pattern. The invention is also directed to methods for quantifying blood glucose levels using the inventive optical device and manufacturing methods for disposing the glucose-sensing coating onto, or incorporating it into, the contact lens material.

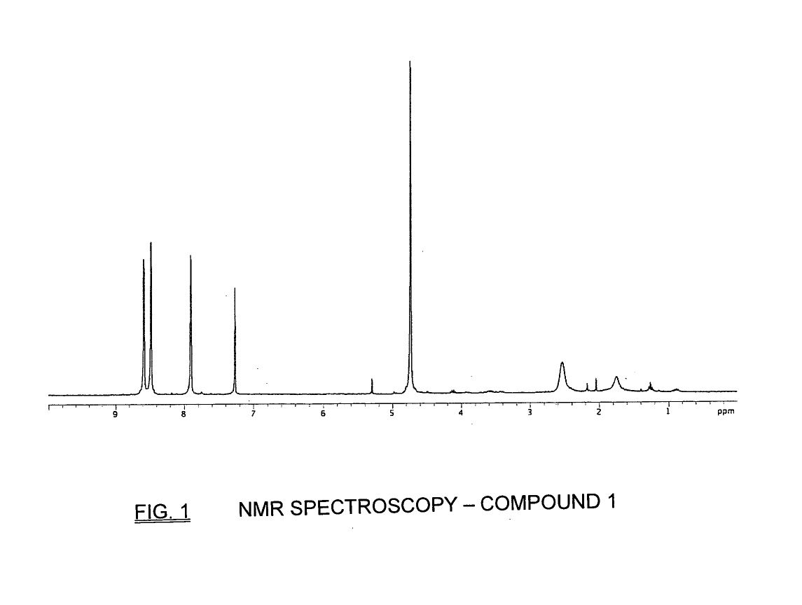

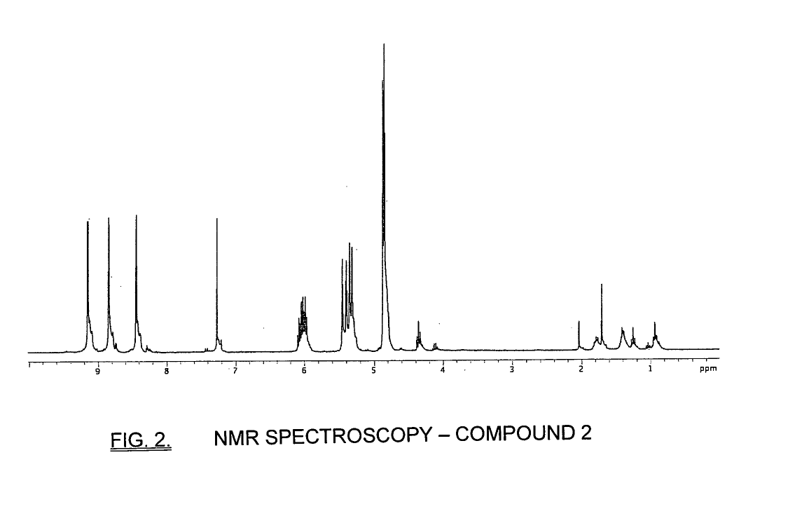

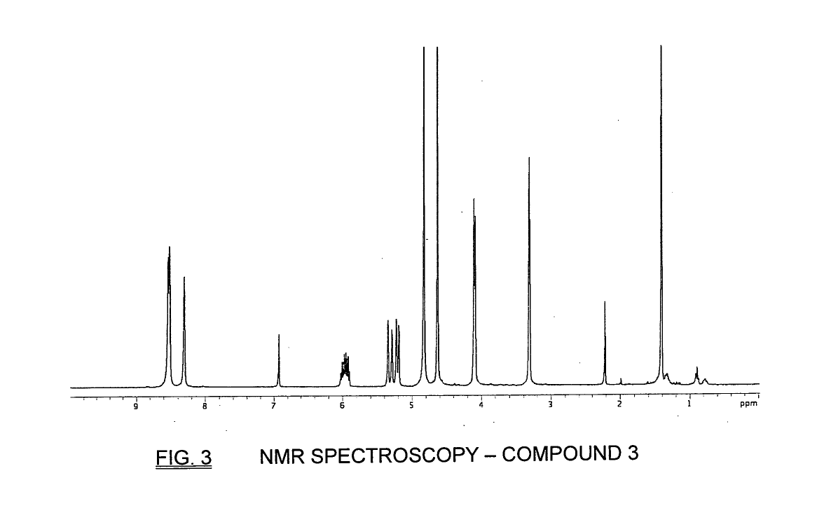

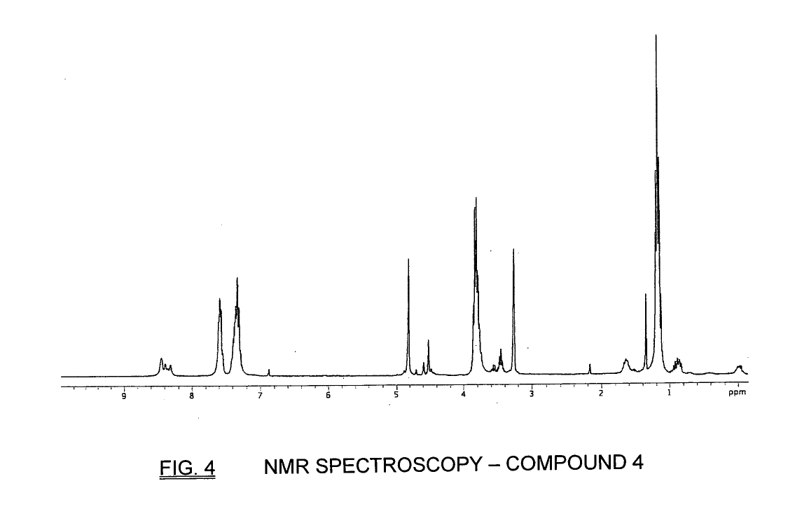

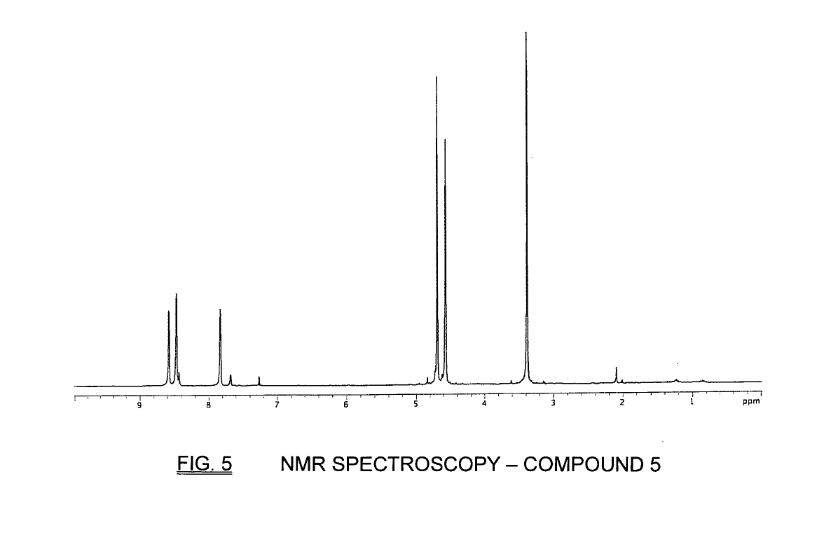

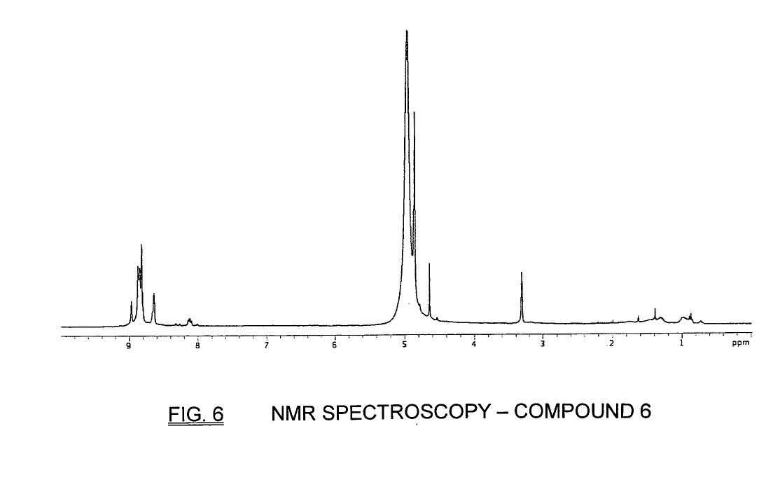

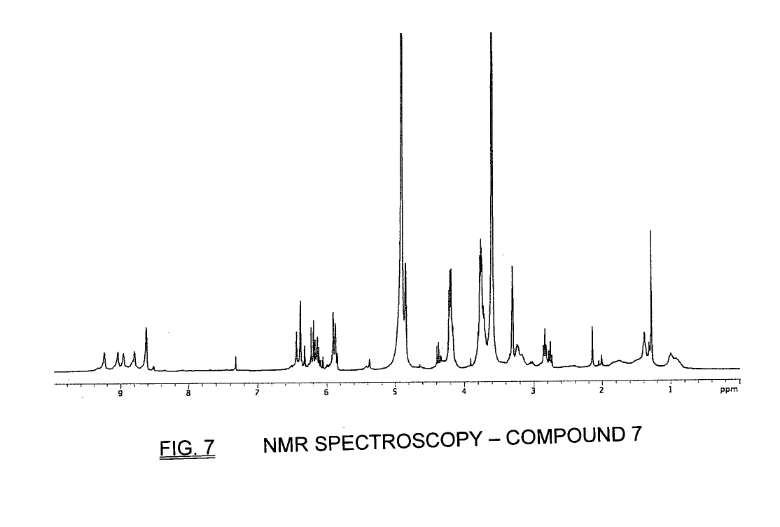

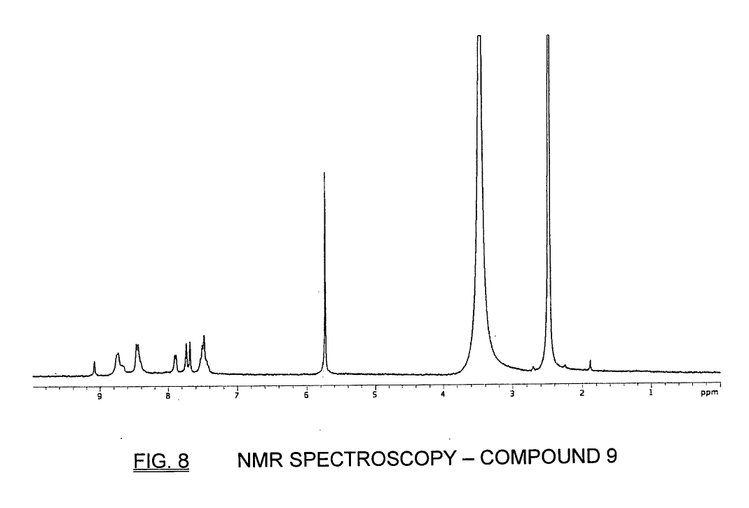

### FIELD OF THE INVENTION

This invention is directed to a device and method for non-invasive real-time testing of blood sugar levels in a diabetic patient. Specifically, this invention is directed to an optical device comprising a contact lens having a glucose-sensing optical pattern imprinted, marked, coated or otherwise disposed on or incorporated within the contact lens. The indicator pattern is further comprised of a glucose-sensing coating containing a boronic acid derivative, which reacts in the presence of glucose to create a readable pattern, which can then be correlated to a pre-determined or pre-calibrated blood glucose level. A polarized light source is one method that may be used to read the indicator pattern. The invention is also directed to methods for quantifying blood glucose levels using the inventive optical device and manufacturing methods for disposing the glucose-sensing coating onto, or incorporating it into, the contact lens material.

### BACKGROUND OF THE INVENTION

Glucose sensors have long been the subject of studies due to their importance in the diagnosis and treatment of diabetes. The International Diabetes Federation recently reported that there are over 177 million diabetics worldwide with the potential of a dramatic increase in that number in developing countries. Moreover, obesity is an ever-increasing public health problem. Diabetes is considered to be the prime medical complication in patients who are overweight. Diabetes is also a risk factor for cardiovascular or cerebrovascular disease. Hence, monitoring of blood glucose levels in diabetes is implicated in a number of co-morbid states.

Hand-held electrochemical glucose-sensing devices, or glucometers, are now in clinical use by diabetic patients for monitoring blood glucose levels. These glucometers utilize a strip, comprising an electrode, upon which a blood sample is placed. The electrode comprises, among other things, a glucose oxidoreductase enzyme. Glucose detection is based upon oxidation of glucose catalyzed by the glucose oxidoreductase enzyme. Upon exposure to a blood sample, the electrode detects the electrons generated in the reaction between glucose and the enzyme through an electron coupler, such as ferrocene, that is also bound to the electrode surface. Depending on the concentration of glucose in the sample, more or less electrons are generated. The number of electrons generated is converted to a numerical readout of glucose concentration.

Glucometers provide convenient one-shot measurements of blood glucose using a blood sample obtained through a pinprick to a finger or the arm. The successful development and commercialization of these electrochemical glucose sensors have provided diabetic patients with essential means for monitoring and self-management of their chronic disease state.

Notwithstanding, glucometers are not without disadvantages. Many diabetics complain of the pain associated with repeated pinpricks necessitated by frequent monitoring schedules. Most conventional meters need to be calibrated each time a new supply of strips is purchased. Moreover, strips are specifically designed for their respective meters, are usable one-time only, and are quite costly. Even in so-called “self-calibrating” or “no calibration” meters, specific strips must be utilized. Strips have a limited shelf life, and the meter will not function if the expiration date of the strips is exceeded.

A further advantage is that results obtained are not always reliable and are heavily influenced by blood sampling technique. This is especially important in the elderly or handicapped, who may not have the manual dexterity to manipulate the strip and meter or to obtain an appropriate sample.

The art has recognized a need for accurate, reliable minimally invasive techniques to analyze blood sugar with minimal time between sample taking and read out, without the above-noted disadvantages associated with glucometers. “Gluco Watch”, which is based upon iontophoretic extraction of body fluid through skin, is one method that has been developed for minimally invasive monitoring of blood glucose. While there is less discomfort than with traditional glucometer use, this device still has a significant time delay between obtaining the sample and obtaining a blood glucose concentration readout. The method also suffers from several calibration disadvantages.

Other strategies are currently under investigation for non-invasive glucose monitoring, including the use of near-infared (NIR) spectroscopy and implantable sensors. The goals of these strategies are to minimize discomfort and cost associated with traditional methods and allow for “real-time” monitoring, or very minimal time between sample taking and readout. Thus far, these goals have not been realized.

“Real-time” in vivo monitoring of analytes, such as glucose, in critical-care patients remains a long-standing and elusive goal in biosensor design and fabrication. The development of long-term implantable glucose sensors, suitable for minimally invasive or non-invasive repeated real-time detections, has not been achieved despite a tremendous amount of research. One of the main problems is that the research, thus far, is based upon the reaction of glucose with an enzyme. The main difficulties encountered with this approach are short half-life of the enzymes used to react with glucose, complications from enzyme co-factors and bio-incompatibility of the sensing interfaces with the body. The high cost of fabrication and the complexity of calibration render the mass production of these implantable sensors difficult. In addition, biosensors made of enzymes and other biomaterials are usually not compatible with the common sterilization methods required for in vivo applications.

Glucose sensing and sugar analysis in biological fluids thus remain a “Holy Grail” in bioanalytical science. Sugar molecules usually display very low optical densities and spectroscopic signatures in aqueous solutions. Direct spectroscopic measurements are also complicated by peak broadening due to the strong hydrogen bonds and conformation changes in aqueous solutions. “Real-time” analysis has not been achieved.

Alternative research into glucose analysis is ongoing. Over the past decade, much research has been devoted to electron transfer fluorescence quenching sensors for glucose analysis, based upon benzylaminoboronic acid. This method suffers from two chemical structural difficulties. First, the energy of the emitting fluorophores must match that of the non-bonding electrons of the amino group for electron transfer fluorescence quenching. This requires that the excitation light be at a UV wavelength where biological molecules also absorb and fluorescence. Second, benzylboronic acids usually bind to glucose at above pH 8. At physiological pH, protonation at the amino group occurs to compete with boron coordination for binding to glucose, thus making this approach non-feasible.

As another alternative to the enzymatic reaction-based sensing methods discussed above, affinity sensing (or binding) utilizing synthetic “receptors” as spectroscopic transducer units is considered a promising “implantable” approach. As in receptor-ligand or antibody-antigen interactions, molecular recognition processes associated with this type of sensing mechanism involve no chemical reactions, and the difficulties in quantifying enzyme cofactor effects on reaction rates are, therefore, eliminated. Affinity binding is also one of the most widely applicable mechanisms of sensor design that allows for relatively easy coupling with optical and electronic detecting methods.

In developing affinity-based glucose sensors, it is important to have a viable, accurate molecular recognition technique. Reversible covalent complexation between phenylboronic acid and diols is one such technique that has been studied extensively, especially for glucose sensors. (Other commonly used molecular recognition techniques, such as hydrogen-bonding interactions are usually ineffective in these conditions.)

Glucose exists in two basic structures—straight chain and ring. The ring structure predominates in more than 99% of circumstances. There are two forms of the ring structure: α-glucose and β-glucose. These two forms interconvert and exist in equilibrium when glucose is dissolved in water. Specifically, in aqueous solution, glucose interconverts to several structural forms, including α-D-glucopyranose, β-D-glucopyranose, α-D-glucofuranose, and β-D-glucofuranose. These structures have 1,2-diol binding sites that can form reversible covalent bonds/complex with boronic acids to form boronic esters. Because of the rapid structural interconversions of glucose and the reversibility of the glucose/boronic acid complex, glucose, boronate, boronic esters and other acid-base species form complex equilibriums in an aqueous solution.

It was found that under the conditions of normal physiological pH and blood glucose concentrations, most of the glucose molecules and boronic acid are not bonded because their bimolecular association constants are too small (less than 15 even with organic solvents such as methanol as a co-solvent). Hence, the potential of using boronic acid for glucose sensing applications is hampered by these typical low bimolecular binding isotherms. In short, the bonding strength is insufficient to withstand small perturbations in chemical (such as pH) and physical (such as temperature) conditions to be useful for physiological sensings.

To achieve the necessary selectivity and specificity for glucose sensing applications for diabetic care, it is necessary to have boronic acids with bonding affinities similar to that of polyclonal antibodies. Boronic acids with high glucose binding affinities have been sought. Most research efforts were devoted to the use of bis- and multi-boronic acid scaffolds (molecular structures) to achieve recognition and necessary chelating binding of substrates such as glucose. In reported favorable cases, the intrinsic selectivity and sensitivity of properly-spaced boronic acids on appropriate scaffolds rivals that of an enzyme-based sensing method due to the chelating effects of bidentate and multidentate bindings. Polymer-based boronates have also been developed for sugar complexations, showing comparable results.

At the University of Akron, it was first discovered that the bimolecular binding for glucose of an aromatic boronic acid is dramatically greater when a nitrogen atom is incorporate directly into the aromatic ring bearing the boron. At physiological pH, this nitrogen atom is protonated in aqueous solution, which causes the boronic acid site to be triol binding to form a more stable zwitterionic complex with glucose. In particular, 3-pyridinylboronic acid, a zwitterionic arylboronic acid, was found to bind glucose at the 3, 5, 6-triol of glucose, which forces the glucose to adopt predominantly the α-D-glucofuranose form. This allows both of the 1,2-diols of the α-D-glucofuranose to be axial, facilitating the specific tight binding to another such boronic acid of a comparable binding isotherm. Therefore, 3-pyridinylboronic acid typically forms a 2:1 complex with glucose (in mM concentrations) under physiological conditions. This discovery is remarkable and important for the development of new materials useful for the contact lens glucose sensors described herein.

The design and enabling experiments for using an arylboronic acid-based molecular sensor for glucose in diabetic monitoring in conjunction with a microscopic, non-enzymatic, implantable sensor(s), which can be optically read and which comprises polymer-encapsulated pyridinylboronic acid and derivatives have been described in WO2006/050164 incorporated herein by reference. Briefly, an implantable polymer capsule was designed to be biocompatible or biodegradable in the human body. Non-invasive colorimetric and Raman spectroscopic read outs of the reversible binding reactions of the implanted sensors were demonstrated. This permitted the use of chemical enhancement agents for in vivo sensing and molecular imaging using Raman spectroscopy/spectromicroscopy.

While demonstrating a significant advancement, these implantable sensors are not practical from a day-to-day monitoring perspective. Raman spectroscopy and other optical readout approaches are not readily available in most settings. The implanted sensors themselves may bd rejected, cause some irritation, or be prone to malfunction. Overall, implantable sensors and Raman spectroscopy are quite costly. There remains, therefore, a need for an affordable, accessible, reliable and accurate method to detect blood glucose, which is also non-invasive and approximates “real-time” values.

For diabetics, adherence to a routine schedule of glucose monitoring and self-management is important. Tight control of blood sugar is associated with decreased occurrence of co-morbidities in a diabetic patent. In addition, the prognosis for patients suffering from diabetes and its complications can be substantially improved, if the condition can be detected earlier and easier and if blood glucose can be monitored on a day-to-day basis at minimal patient discomfort and cost. Significant advantages could be gained if a non-invasive and affordable method was available, so that the patient's blood sugar can be more frequently monitored and tightly controlled over time, ideally by “real-time” monitoring methods, without the attendant disadvantages of other methods discussed above.

Recent studies have shown that human tears contain about 10-15% of blood sugar (plasma glucose), with a latency of about 20 minutes from blood values. Tears are interstitial fluids. Concentrations of glucose in interstitial fluids usually follow and correlate well with that in plasma under specific physiological conditions by the diffusion limiting equilibrium. The well-defined diffusion profile of tear glands and rich micro-circulation surrounding the eyes result in reliable correlations of glucose concentrations between the plasma and tears with almost no delay time. It is, therefore, feasible to monitor blood, sugar (plasma glucose) indirectly from tears with non-invasive sampling techniques. From a clinical point of view, glucose concentrations in tears can be used to monitor blood glucose of diabetic patients with the same efficacy as conventional blood sugar monitoring where blood is drawn directly from a fresh pinprick to a finger or arm.

The present invention describes a new technique for monitoring glucose in tears with an optical device that patients can wear in their eyes. One embodiment is a soft contact lens incorporating a glucose-sensing coating material that is stamped, imprinted, marked, or otherwise applied to or disposed on the contact lens surface, or imbedded or layered or otherwise incorporated within the contact lens, in a pattern. Upon exposure to glucose, the coating material molecules change their optical properties through mesogenic reorientation, and the pattern becomes readable through one or more methods. In one such method, glucose concentration levels in the blood can be observed by the patients in real-time using a simple technique, such as a polarizing light source.

The glucose-sensing coating material is designed to achieve high selectivity and accuracy. This approach represents a new totally non-invasive device and method for sensing and monitoring blood glucose in a diabetic patient. Calibration can be achieved by varying the concentration of glucose-sensing molecules in the coating material. While calibrating is not necessary, if there is any question about reliability based upon patient-specific factors, such as anatomy, circulatory problems, tear volume and the like, the device can be calibrated or checked by patients using the conventional, pinpricking plasma sugar sampling technique and related electronic glucometers. The number of painful pinpricking procedures can be greatly reduced, however, without sacrificing the sensing accuracy and, hence, achieves high patient compliance to a tight monitoring regimen.

The invention is also directed to manufacturing methods for incorporating the glucose-sensing coatings of the invention into typical hydrogel contact lens material, using molding technology.

While the invention is conducive to non-invasive monitoring of blood glucose directly by diabetic patients using simple polarizing light devices, the invention's optical devices may also be used in conjunction with imaging devices, such as cameras, which, upon sensing the change in the optical pattern in response to glucose, can provide automated numerical readouts useful for monitoring glucose levels. These readouts can be used not only for routine monitoring, but also for warning if blood sugar levels become too high or too low. They may also be used as closed-loop sensors for devices, such as an artificial pancreas or an insulin pump, which helps to regulate insulin release and, hence, blood glucose within normal physiological limits.

### SUMMARY OF THE INVENTION

This invention is directed to the design and manufacturing of glucose-sensing optical coatings capable of being used in the eye, the use of such coatings in the design of a glucose-sensing contact lens (or other ocular inserts) and methods for monitoring and quantifying results, and clinical implementation of non-invasive, real-time blood glucose concentration monitoring methods, based on tears.

In one embodiment, the invention is directed to glucose-sensing coatings comprising 3-pyridinylboronic acid, substituted pyridinylboronic acid derivatives, or mixtures thereof, in combination with polymeric materials, including without limitation polymers having various morphologies, or with lyotropic liquid crystal materials.

In another embodiment, the invention is directed to a contact lens having disposed on its surface, imbedded within the lens, or layered between the contact lens material, a pattern formed from the glucose-sensing coating.

In still another embodiment, the invention is directed to a method of monitoring blood glucose wherein the coating disposed on the contact lens interacts with blood glucose resulting in a pattern that is then read using a polarized light source.

In yet another embodiment, a manufacturing method for incorporating glucose-sensing optical coatings into contact lens material is described.

Finally, in addition to readouts using polarized light sources, this invention may be used with other devices, such as an imaging camera, which can provide automated numerical readouts, which, in turn, can be used as feedback to regulate other devices.

### DETAILED DESCRIPTION OF THE INVENTION

Glucose-sensing optical coatings utilizing an affinity-based glucose sensing mechanism, rather than an enzyme-based sensing mechanism, have been developed. These coatings are based on 3-pyridinylboronic acid and related structures or substituted pyridinylboronic acids and derivatives, which can then be combined with (disposed on or incorporated within or into) existing soft contact lens materials. The coatings utilize polymers and/or liquid crystals having various morphologies, including among other things linear, branched, star, comb, dendritic and nanoparticle structures. These new engineering coating materials can self-assemble into sheets, cylinders, and other supramolecular assemblies, as well as with functionalized metal (gold) nanoparticles and nanorods. They can be large or small molecules. They must be compatible with contact lens materials.

Structural examples of coatings that may be designed using polymers, such a hydrogels, dendrimers or nanoparticles in combination with the aforenoted boronic acids are shown below.

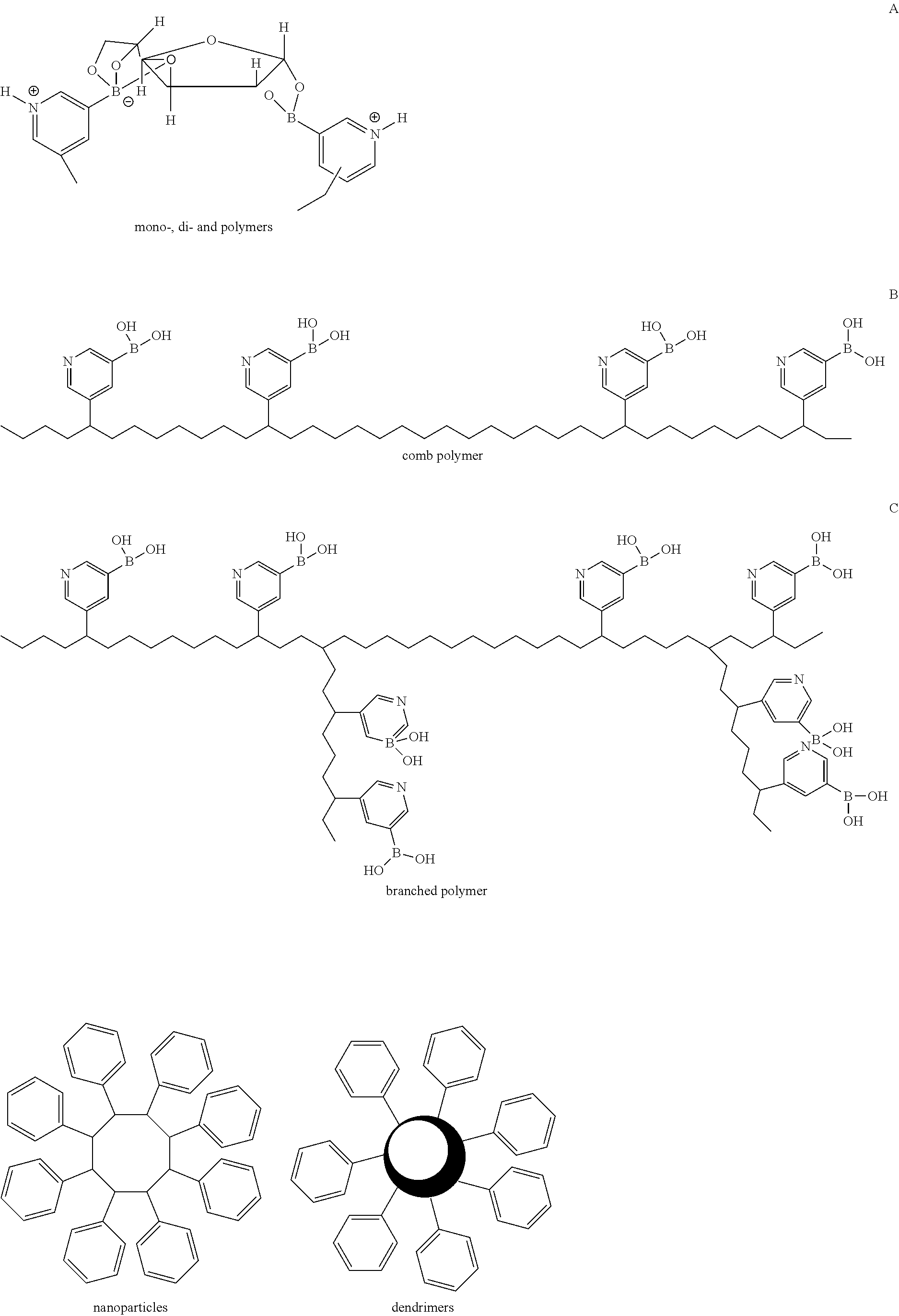

The following structures illustrate inventive optical coatings based upon liquid crystals.

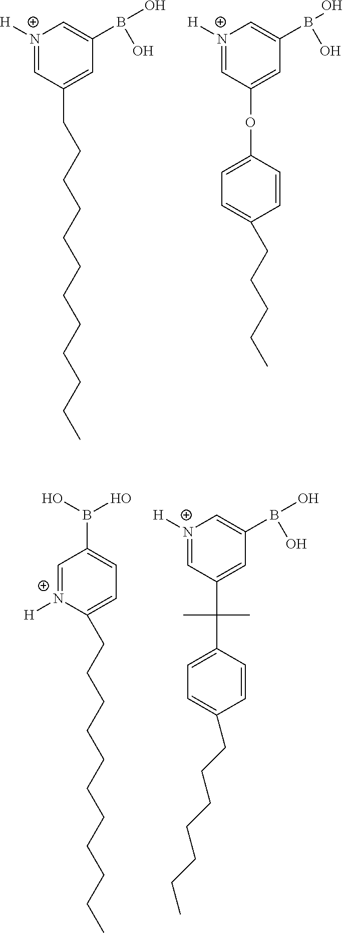

The optical coatings of the invention are designed such that when glucose concentration increases in the media of interest, specifically blood, cross-linking of the glucose-sensing materials, such as the 3-pyridinylboronic acid moieties, in the coating increases. When glucose concentration decreases, crosslinking decreases. The unique binding events between the sensing component (coating) and glucose result in mesogenic reorientation of the optical properties of coatings specific to (and quantitative of) the glucose concentration. The concept is very similar to a typical LCD display, wherein the optical properties of a thin film are controlled by applied voltages. Here, the optical properties are controlled by glucose binding events. Glucose is optically active. However, the effect is very small by itself. The mesogenic materials are used to amplify the small differences in gluocose concentration through superamolecular ordering/phase transitions within the coatings in direct response to the concentration.

In one embodiment, the glucose-sensing contact lens of the invention is a typical contact lens, that has been imprinted, marked or coated with, or otherwise having applied or disposed on, the optical coatings discussed above. The coatings may also be imbedded in or layered between the contact lens material. Techniques for incorporating the coatings onto or within a contact lens are described below. These techniques are not meant to be exhaustive.

In another embodiment, a contact lens or other ocular insert is imprinted with a latent, optically active glucose concentration scale image or pattern, comprising the aforenoted coatings, on or within the lens. The pattern is designed with easily readable optical directions, and the lens is produced to minimize free rotations in the eye when wearing. The contact lens or insert is otherwise optically identical to a typical contact lens, and the glucose concentration scale image is invisible with isotropic light sources. Upon exposure to glucose, the glucose-sensing materials reorient to create a pattern that is visible using polarized light. With a linear polarizer in hand or the use of a pair of polarized glasses, which convert natural light into polarized light, the patient can see the optical pattern created by the reaction of the coating with glucose. The pattern can be calibrated to display quantitatively the blood sugar level at any time, without drawing blood.

The coatings are applied to otherwise disposed on the surface of the contact lens in any optical pattern that can be discerned easily by the user with a polarized light source. Alternatively, the coatings may be imbedded or layered in a pattern within the contact lens material during manufacturing of the lens itself.

Clinically, in use, the optical patterns cannot be sensed in the absence of glucose. The presence of glucose induces mesotropic or chiral mesotropic orderings in the coating molecules that change the polarization of the light. By varying the concentration of the glucose-sensing coatings, phase transitions can be quantitatively controlled to reflect the concentration of glucose in the tears and, hence, the blood. The readings approximate real time, since there is little delay in the presence of glucose in the tears after it is present in the blood. The quantitative scale is controlled by the concentration of glucose binding sites incorporated in the coating materials and other materials properties, which are calibrated and set during manufacturing.

As with most contact lenses, the inventive glucose-sensing contact lens is disposable after a certain time, usually a week.

Patients wearing the imprinted contact lens are able to read the patterns in the contact lens, using a simple, linear polarized light device. A hand held polarizer or polarized glasses provide a linear polarized light source from readily available natural light. Without a polarizing light source, the contact lens' glucose-sensing pattern cannot be seen. With a polarizing light source, the patient can see the glucose-induced patterns in the lens.

As discussed above, the inventive contact lens can be pre-calibrated to meet specific diabetic needs, correlating specific glucose values with discernable patterns. For example, for a patient with high blood sugar levels, the dynamic range of the device can be adjusted to be more sensitive for higher blood glucose levels thus assuring that the pattern is most visible for higher values. Similarly, the range of the device can be adjusted to be less sensitive to normal physiological levels of glucose. The range of the device may also be adjusted to reflect low blood glucose values as well, in a patient prone to hypoglycemia. Patients can further calibrate or check the contact lens readings using a conventional glucometer, if desired.

Techniques for applying or incorporating the glucose-sensing optical coatings to contact lens material include in situ photo polymerization, micro-injection and ink jet printing. Other methods known to those skilled in the art may be used.

Typical soft contact lenses are made of hydrogels, such as poly(hydroxy-ethyl methacrylate) and poly(ethylene oxide)-co-polysiloxide. The inventive optical coatings are water soluble and compatible with both of these materials. Other conventional contact lens materials are known to those skilled in the art and are considered within the scope of the invention.

Control of the shape and color patterning of contact lenses is well established using current injection molding technology. In injection molding, the contact lens polymer material is injected into the mold under pressure and cured/crosslinked thermally or with radiation. The lens is then removed from the mold and finished on a lathe. Lenses may also be produced entirely through molding, that is, they need no lathe cutting. This is a recent development, made possible through highly automated, computer-controlled mold production.

One manufacturing method for incorporating the inventive glucose-sensing optical coatings into contact lens material to produce glucose-sensing optical devices utilizes conventional molding technology. To produce the optical pattern in the contact lens, a two-step molding method is utilized to allow encapsulation of the glucose-sensing optical coatings in the contact lens so that they do not directly interact with the eyes when in use. In the first step, a thin layer of the contact lens polymer material is spin-coated in a mold and partially cured. The optical pattern is formed on the first layer by screen or ink-jet printing. A second layer of the contact lens polymer material is then injected into the mold and finally cured to form the glucose-sensing contact lens or ocular insert.

More advanced patterning and imprinting techniques allowing for mesotropic orientation of the glucose-sensing coating pattern in a more precise way, so that quantifications can be performed easily, may also be used. For example, photopolymerization methods may be applied in manufacturing the glucose-sensing contact lenses, although ink-jet or screen-printing methods are more cost effective and allow for a mass production method. Other methods known to those skilled in the art may be used to apply the glucose-sensing coating materials to the surface of the lens or within the contact lens. All these methods are compatible with the current manufacturing and sterilization methods for contact lens and, thus, little regulatory inhibition is expected.

Although it is contemplated that the inventive devices will be most useful in monitoring blood glucose levels by diabetic patients using simple light-polarizing devices, the invention is not limited to such applications. It is contemplated that the inventive optical devices may be utilized in conjunction with other reading devices, such as an imaging camera, which can be used to generate automated numerical readouts for monitoring glucose levels, including for warnings if glucose levels become too high or too low, and as closed-loop sensors for regulating other devices. Specifically, in one embodiment, the glucose-sensing optical pattern of the contact lens (or other ocular insert) is “machine readable” with a common digital camera. The images are computer-analyzed to provide quantitative readings of the glucose concentration within seconds of reading. The imaging device can be further used as an automatic reader allowing glucose concentrations to be monitored around the clock, providing warning signals if levels become too high or too low, requiring a clinical intervention. The automated readout mechanism can also be used as a feedback for an insulin pump, allowing blood sugar monitoring and regulation of insulin levels to be carried out in tandem, using the same device as is used to close the loop for precise control of blood sugar levels with an artificial pancreas, for example.

### EXAMPLES

Three exemplary types of materials for the inventive coatings have been designed and are depicted herein:

(1) Helical polymers, wherein a linear, semi-stiff polymer is produced with a preference of one helical orientation, for example, M-helix. Upon glucose binding, the orientation switches to P-helix, which changes the optical rotation of the material;

(2) Comb polymer liquid crystals with glucose binding sites distributed in the side chains. Upon glucose binding, which form rigid 1:2 complexes with boronic acids, the comb polymer liquid crystals change optical orientations due to the scaffolding effect of the chirality of the complexes.

(3) Discotic liquid crystals with glucose binding sites distributed in the peripherals of the disks. Glucose binding changes the optical rotation of the film.

It is intended that all of the inventive optical coatings are polled or otherwise designed to produce a defined linear polarization directly in the film upon exposure to glucose. The transitions can be induced by changes in glucose concentration, thus facilitating glucose read outs.

Example 1

### Helical Polymer such as Polyisocyanates and Polyamides

### Example 2

### Side Chain Liquid Crystals (Comb Polymer Liquid Crystals)

### Example 3

### Discotic Liquid Crystals

### Example 4

### Contact Lens Production

In one method of production, a thin layer of typical contact lens material is spin-coated or otherwise injected or disposed into a mold and partially cured using thermal or radiation curing. Glucose-sensing optical coatings are then formed, imprinted, marked, or otherwise disposed on the partially cured layer in a pattern using screen or ink-jet printing. A second layer of contact lens material is then injected into the mold over the glucose-sensing pattern. Final curing forms the contact lens with the glucose-sensing optical pattern layered within the lens.

Examples 5 and 6 reflect synthesis of biocompatible hydrogel monomers useful in the practice of the invention.

### Example 5

### Cyclic Siloxane

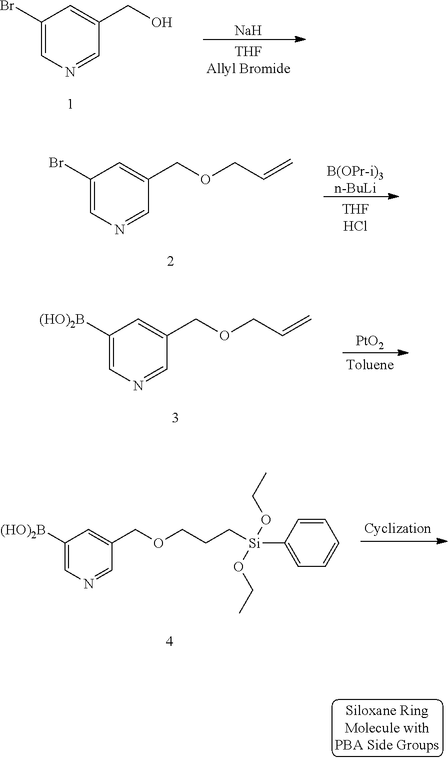

The components utilized in the synthesis of the cyclic siloxane are numbered as above. Methods of production for the components are described below. Each “compound” corresponds to the number in the above synthesis sequence.

Compound 1 was synthesized following the reported procedures as exemplified by the following references: Bachman, G. B.; Micucci, D. D. J. Am. Chem. Soc., 1948, 70, 2381-2384 and Zhang, N.; Tomizawa, M.; Casida, J. E. J. Med. Chem. 2002, 45, 2832-2840.

Compound 2

To a THF solution of NaH and compound 1 (1 g), a solution of allyl bromide in THF (10 ml) was added slowly. Then the mixture was heated to reflux for 20 hours. The reaction was quenched with 15 ml of water. The organic layer was separated, and the aqueous layer was extracted with THF (20 ml×2). The organic layer was combined and concentrated. Pure product was obtained as a colorless oil after column chromatography. (40% EA/Hexanes)

Compound 3

To a 500 ml RBF (flask), 950 mg of compound 2, 50 ml THF and 1.3 ml of B(OPr-i)₃ were added under N₂. The mixture was cooled to −40° C. with a dry-ice/acetone bath. Then 1.2 eq. (equivalents) of n-BuLi was added using a dropping funnel over 40 minutes. The mixture was stirred for another 40 minutes under −40° C. After that, the dry-ice/acetone bath was removed. 35 ml of HCl was added while it reached −20° C. After the mixture reached room temperature (RT), it was transferred to a separating funnel. pH was adjusted to 7˜8 with 5 N of NaOH solution. Then, it was extracted with THF twice. The organic layers were combined and concentrated.

Compound 4

A solution of compound 3 (850 mg) in toluene was heated to 110° C. for 10 hours to eliminate water with a dean-stark trap. Then, 1.1 eq. (951 mg) of diethoxy phenylsilane was added, followed by platinum oxide. The mixture was stirred at 78° C. for overnight. The reaction was not complete until reacted at 100° C. for two days.

### Example 6

### The Following Product was Synthesized

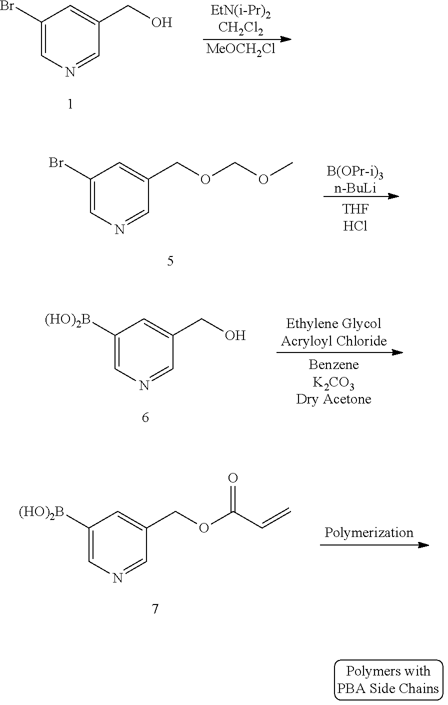

The components utilized in the above synthesis are numbered as above. Methods of production for the components are described below. Each “compound” corresponds to the number in the above synthesis sequence.

Compound 1 was synthesized as described in Example 5.

Compound 5

To a two-neck RBF, 1.3 g of compound 1 was added, followed by 9 ml of EtN(iPr)₂. The mixture was cooled down to 0° C. with an ice bath. 1.3 ml of chloromethyl methyl ether was added dropwise with a syringe. 10 ml of CH₂Cl₂ was added to help dissolving the salt precipitate. The mixture was stirred for 1.5 hours at 0° C. and then for 16 hours at room temperature (RT). The reaction was quenched with a 50 ml solution of saturated NH₄Cl and ammonia (1:1). Then it was extracted with ether twice. Pure product was obtained as a colorless oil after column chromatography. (50% EA/Hexanes).

Compound 6

To a 500 mL RBF 1.02 g of compound 5 and 40 mL THF were added under N₂. The mixture was cooled to −40° C. with a dry-ice/acetone bath. Then, 1.2 eq. (equivalents) of n-BuLi was added using a dropping funnel over 40 minutes, followed by 1.35 ml of B(OPr-i)₃. The mixture was stirred for another 40 minutes under −40° C. After that, the dry-ice/acetone bath was removed. 35 ml of HCl was added while the mixture reached −20° C. After the mixture reached room temperature, it was transferred to a separatory funnel. pH was adjusted to 7˜8 with 5 N NaOH solution. Then it was extracted with THF twice. The organic layers were combined and concentrated.

Compound 7

230 mg of compound 6 was dissolved in 30 ml of benzene, followed by addition of 110 mg of ethylene glycol. The mixture was heated to reflux overnight. Then it was cooled down to RT. 5 ml of dry acetone was added, followed by 1.5 g of K₂CO₃ and 400 mg of acryloyl chloride. The mixture was stirred at RT overnight. The product was extracted with CH2Cl2 from water, then concentrated with rotavapor.

### Example 7

### Glucose Sensing Liquid Crystal

One embodiment of the inventive glucose sensing compositions and a method for preparation is described below.

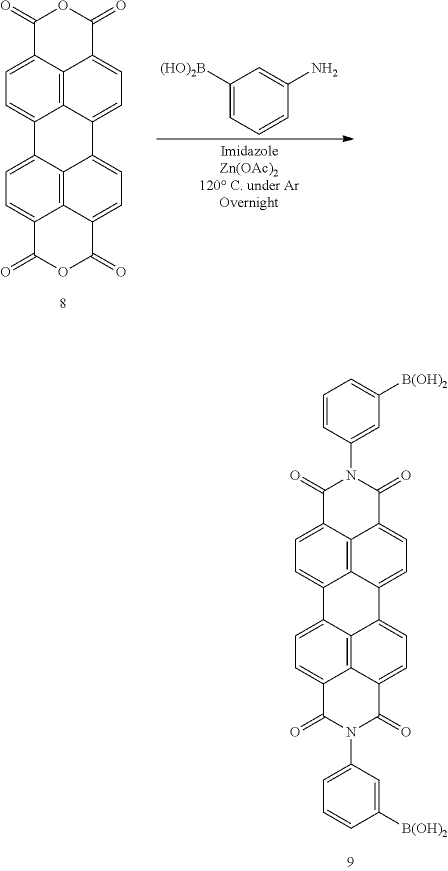

Compound 8 (3,4,9,10-perylene tetra-carboxylic dianhydride)(CAS Reg. No. 128-69-8) and 3-aminophenylboronic acid were purchased from Acros and used as received without further purifications.

Compound 9

To a two-necked RBF, 313 mg (0.8 mmol) of compound 8 and 250 mg (1.6 mmol) of 3-aminophenyl boronic acid were added, followed by addition of 3 g of imidazole, 14 mg of Zn(OAc)₂·2H₂O. The mixture was heated under argon at 120° C. overnight. The solid was dispersed in 100 ml of ethanol, followed by addition of 50 ml of concentrated HCl and 250 ml of water. The mixture was stirred for 24 hours. Then it was filtered through a membrane filter and washed thoroughly with water, yielding a dark-red solid as product.

In accordance with the patent statutes, the best mode and preferred embodiment have been set forth; the scope of the invention is not limited thereto, but rather by the scope of the attached claims.

What is claimed is:

1. A glucose-sensing coating disposed on or incorporated within a contact lens or ocular insert, comprising:
3-pyridinylboronic acid, substituted pyridinylboronic acid derivatives, or mixtures thereof; and
a polymer or liquid crystal, wherein the polymer or liquid crystal is compatible with conventional contact lens materials.

2. The coating as set forth in claim 1, wherein the polymer comprises a linear, branched, star, comb, or dendritic polymer; or self-assembled nanoparticles; or mixtures thereof.

3. The coating as set forth in claim 1, wherein the polymer comprises polyisocyanates, polyamides, silicon-based polymers, comb polymer liquid crystals, or discotic liquid crystals, or mixtures thereof.

4. The coating as set forth in claim 2, wherein the nanoparticles are metallic and comprise silver or gold, or mixtures thereof.

5. A device for determining blood glucose levels, comprising:
a contact lens having disposed on its surface, or imbedded or layered within, a glucose-sensing coating comprising;
3-pyridinylboronic acid, substituted pyridinylboronic acid derivatives, or mixtures thereof in combination with a polymer or liquid crystal material;
wherein the coating is disposed on the contact lens surface, or imbedded or layered within the contact lens, in an optical pattern;
wherein the pattern changes in response to glucose present in tears; and
wherein the pattern is read by the use of a readily available, polarizing light source.

6. A method of determining blood glucose, comprising:
placing in the eye a contact lens, having a glucose-sensing coating disposed on a surface of the contact lens, or imbedded or layered within the lens, in a pattern, wherein the coating comprises 3-pyridinylboronic acid, substituted pyridinylboronic acid derivatives, or mixtures thereof, in combination with a polymer or a liquid crystal material;
providing a source of polarized light; and
reading the pattern resulting from an interaction between glucose in tears and the glucose-sensing coating; and
correlating the pattern with a pre-calibrated glucose level.

7. A method of manufacturing a glucose-sensing optical device, comprising the steps of:
providing a contact lens material into a mold;
partially curing the material to form a first layer;
forming an optical pattern on the first layer using a glucose-sensing optical coating;
injecting a second layer of contact lens material into the mold over the optical pattern; and
curing.

8. A method for monitoring blood glucose levels, comprising:
providing an optical device having a glucose-sensing optical coating disposed thereon in a pattern;
utilizing an imaging device to read changes in the optical coating pattern in response to glucose levels; and
correlating the readout from the imagining device to a pre-determined glucose level.

9. A method as set forth in claim 8, further comprising:
utilizing the readout from the imaging device as a closed loop sensor for other devices such as an insulin pump or artificial pancreas.
